(19) United States
(12) Patent Application Publication
(10) Pub. No.: US 2024/0066066 A1
(43) Pub. Date: Feb. 29, 2024

(54) METHOD FOR TREATING ALOPECIA WITH STEM CELL EXOSOME

(71) Applicant: THE FIRST HOSPITAL OF LANZHOU UNIVERSITY, Lanzhou City (CN)

(72) Inventors: Jia YAO, Lanzhou City (CN); Yongcui MAO, Lanzhou City (CN); Pinyan LIU, Lanzhou City (CN)

(21) Appl. No.: 18/047,435

(22) Filed: Oct. 18, 2022

(30) Foreign Application Priority Data
Aug. 24, 2022 (CN) 202211017781.6

(51) Int. Cl. A61K 35/28; A61P 17/14; C12N 5/0775

(52) U.S. Cl. CPC A61K 35/28; A61P 17/14; C12N 5/0665

(57) ABSTRACT

A method for treating alopecia with a stem cell exosome, the alopecia includes androgenetic alopecia, seborrheic alopecia, and post-hair transplantation alopecia. An umbilical cord mesenchymal stem cell exosome has a significant curative effect on androgenetic alopecia, seborrheic alopecia, and post-hair transplantation alopecia.

### CROSS REFERENCE TO RELATED APPLICATION

This patent application claims priority to Chinese Patent Application No. 202211017781.6, filed Aug. 24, 2022, the disclosure of which is incorporated by reference herein in its entirety.

### TECHNICAL FIELD

The present disclosure belongs to the technical field of bioengineering, and in particular relates to a method for treating alopecia with a stem cell exosome.

### BACKGROUND

The rapid development of society has made people's life and work increasingly stressful, resulting in an annually-increased prevalence of androgenetic alopecia (seborrheic alopecia). Alopecia does not affect the physical health, but seriously affects the patient's mental health and living quality. For the alopecia, there are mainly two existing methods for hair growth including drugs and hair transplantation. FDA-approved alopecia drug Rogaine® is currently the only hair growth drug on the market with definite curative effects. However, the drug has a short-term effective dosage, and cannot fundamentally change an internal microenvironment of the scalp. After stopping the administration, the hair growth immediately returns to a state before the administration. Moreover, long-term use of such drug may easily lead to side effects such as hormone level disorders. As another type of treatment, hair transplantation is invasive and expensive; moreover, the hair transplantation does not change a microenvironment of the hair follicles, and only has a temporary medical relief, such that the newly-transplanted hair may be fallen off easily. At present, stem cell exosome-based products are mostly used for repairing damaged tissues and hairdressing, but are rarely used in prevention and treatment of the alopecia. These products are used basically in common seborrheic alopecia.

### SUMMARY

The present disclosure provides a method for treating alopecia with a stem cell exosome. It has been verified by animal tests and clinical trials that the umbilical cord mesenchymal stem cell exosome has a significant curative effect on androgenetic alopecia, seborrheic alopecia, and post-hair transplantation alopecia.

To solve the above technical problems, the present disclosure provides the following technical solutions.

The present disclosure provides a method for treating alopecia with a stem cell exosome, where the alopecia includes androgenetic alopecia, seborrheic alopecia, and post-hair transplantation alopecia. Further, the stem cell exosome may be one or more selected from the group consisting of a human placenta mesenchymal stem cell exosome, a human umbilical cord mesenchymal stem cell exosome, and a human amniotic fluid mesenchymal stem cell exosome, preferably the human umbilical cord mesenchymal stem cell exosome.

Further, a preparation method of the stem cell exosome may include the following steps: culturing mesenchymal stem cells for 24 h to 48 h, and collecting a cell supernatant; conducting centrifugation on the cell supernatant 2 to 4 times, and filtering to obtain a filtrate; conducting centrifugation on the filtrate 2 to 4 times, discarding a pellet I, and conducting centrifugation to collect a pellet II; and resuspending the pellet II with a PBS buffer to obtain a stem cell exosome solution. The mesenchymal stem cells are cultured for 24 h to 48 h with a serum-free basal medium. The mesenchymal stem cells are isolated from the umbilical cord. A medium of the mesenchymal stem cells includes fetal bovine serum (FBS), Dulbecco's modified eagle medium (DMEM), and F12 at a ratio of (0.5-1.5):(3-6):(3-6), preferably the FBS, a DMEM medium, and an F12 medium at a ratio of 1:4.5:4.5.

Further, the centrifugation may be conducted on the cell supernatant by: conducting centrifugation on the cell supernatant at 400 g to 600 g for 5 min to 15 min at 3° C. to 5° C. to obtain a supernatant I; conducting centrifugation on the supernatant I at 1,500 g to 2,500 g for 10 min to 20 min at 3° C. to 5° C. in an EP tube to obtain a supernatant II; and transferring the supernatant II and conducting centrifugation at 8,000 g to 12,000 g for 30 min at 3° C. to 5° C. in an EP tube. The cell supernatant is further filtered after the centrifugation, with a filter membrane at a pore size of preferably 0.1 μm to 0.3 μm. Impurity molecules with different molecular weights are gradually removed by increasing a centrifugal speed step by step.

Further, the centrifugation may be conducted to collect the pellet II by: conducting centrifugation at 300 g to 500 g for 10 min to 20 min at 4° C. to 6° C. to discard a pellet; conducting centrifugation at 2,000 g to 3,000 g for 10 min to 20 min at 4° C. to 6° C. to discard a pellet; conducting centrifugation at 10,000 g to 15,000 g for 25 min to 35 min at 4° C. to 6° C. to discard a pellet; and conducting centrifugation at 125,000 g to 175,000 g for 50 min to 70 min at 4° C. to 6° C. to collect the pellet II.

Further, the pellet II may be resuspended with the PBS buffer by: resuspending the pellet II with the PBS buffer, conducting centrifugation at 125,000 g to 175,000 g for 50 min to 70 min at 4° C. to 6° C., discarding a supernatant to obtain a pellet III, and resuspending the pellet III in the PBS buffer. A crude exosome extract is obtained from the first resuspension.

The present disclosure further provides a stem cell exosome freeze-dried powder, where a preparation method of the stem cell exosome freeze-dried powder includes the following steps: pre-freezing the stem cell exosome solution at −80° C. to −95° C., and vacuum freeze-drying at −40° C. to −60° C. to obtain the stem cell exosome freeze-dried powder. The pre-freezing is conducted at preferably −86° C., and the vacuum freeze-drying is conducted at preferably −50° C. The stem cell exosome freeze-dried powder can be stored at −20° C. for 6 months, with an unchanged biological efficacy.

The present disclosure further provides a stem cell exosome-based thermosensitive gel, including the following components by weight percentage: 0.5% to 1.5% of the stem cell exosome freeze-dried powder according to claims 8, 10% to 30% of poloxamer 407, and 2% to 6% of poloxamer 188. A preparation method of the stem cell exosome-based thermosensitive gel includes the following steps: adding 20% of the poloxamer 407 and 4% of the poloxamer 188 in ultrapure water at room temperature, and allowing to stand for 2 min to 3 min; dissolving the freeze-dried powder in tri-distilled water (at 100 μg/1 ml), adding to a gel matrix at a mass ratio of 1% and fully mixing, and placing in a refrigerator at 4° C. for 24 h to obtain the umbilical cord mesenchymal stem cell exosome-based thermosensitive gel. The well-mixed solution can also be placed at 2° C. to 6° C. for 20 h to 28 h to form a gel system. The stem cell exosome-based thermosensitive gel can be prepared for immediate use, and can be used up within a week. The stem cell exosome-based thermosensitive gel is liquid in the bottle. After being applied to the scalp, due to the influence of skin temperature, the liquid can be solidified into a solid gel within 30 sec; and the solid gel is not easy to fall off when attached to the scalp, such that it is convenient for penetration of the exosome and the active ingredients, thereby increasing a duration of drug action.

In the present disclosure, unless otherwise specified, all raw material components are commercially available products well known to those skilled in the art.

Compared with the prior art, the present disclosure has the following beneficial effects:

In the present disclosure, a hair growth product using the stem cell exosome as an active ingredient can change a microenvironment of the scalp hair follicles, to promote repair and regeneration of the hair follicles and to shorten a resting period of the hair follicles, thereby strengthening hair roots, preventing alopecia, and promoting hair growth. The product has a significant curative effect on the androgenetic alopecia, the seborrheic alopecia, and the post-hair transplantation alopecia.

### BRIEF DESCRIPTION OF THE DRAWINGS

FIG. 1 shows a pellet of an extracted umbilical cord mesenchymal stem cell exosome;

FIG. 2 shows a freeze-dried powder of the umbilical cord mesenchymal stem cell exosome;

FIG. 3 shows an umbilical cord mesenchymal stem cell exosome-based thermosensitive gel;

FIG. 4 shows results of nanoparticle tracking analysis (NTA) identification of the umbilical cord mesenchymal stem cell exosome;

FIG. 5 shows results of Western blot identification of the umbilical cord mesenchymal stem cell exosome;

FIG. 6 shows results of electron microscopy identification of the umbilical cord mesenchymal stem cell exosome;

FIG. 7 shows a hair growth effect of mice in each group;

FIG. 8 shows statistical results of hair diameters of mice in each group;

FIG. 9 shows statistical results of hair length of mice in each group;

FIG. 10 shows statistical results of the number of hair follicles of mice in each group;

FIG. 11 shows results of HE staining of skin tissues of mice in each group; and

FIG. 12 shows an effect of hair regeneration in an alopecia area of a patient.

### DETAILED DESCRIPTION OF THE EMBODIMENTS

The technical solutions in the present disclosure are clearly and completely described below in conjunction with examples of the present disclosure. All other examples obtained by a person of ordinary skill in the art based on the examples of the present disclosure without creative efforts shall fall within the protection scope of the present disclosure.

### Example 1 Preparation of a Human Umbilical Cord Mesenchymal Stem Cell Exosome

1. Isolation of Human Umbilical Cord Mesenchymal Stem Cells
(1) collection of human umbilical cord: the human umbilical cord was placed in an umbilical cord storage bottle through aseptic operation, and immediately transferred to a laboratory for treatment at a constant temperature of 4° C.;
(2) alcohol disinfection: the human umbilical cord was placed in a beaker to conduct disinfection with 75% alcohol by submerging the umbilical cord for 10 sec to 12 sec, the umbilical cord was transferred to another beaker containing 50 ml of a normal saline and rinsed repeatedly 2 to 3 times, the liquid was discarded;
(3) removal of congestion: the umbilical cord was cut in a flat dish containing 50 ml of the normal saline using surgical scissors and hemostatic forceps, to obtain 5 to 10 segments with a length of 0.5 cm to 1 cm, congestion was removed, and the excess umbilical cord was put back into the umbilical cord storage bottle for later use; the cut umbilical cord was put into a sterile beaker with the hemostatic forceps, washed with the normal saline 2 to 3 times, and the washing solution was discarded;
(4) cutting into pieces: the cleaned umbilical cord was placed in a sterile beaker, and the umbilical cord was cut into small pieces using the sterile surgical scissors, with a size of 2 mm³ for each piece;
(5) inoculation: shredded umbilical cord tissue blocks were evenly inoculated into at least 3 75 T culture bottles with a sterile long spoon, with greater than or equal to 200 blocks in each bottle;
(6) drying: the 75 T culture bottles with the umbilical cord tissue blocks were put into a 5% CO₂, saturated humidity, and 37° C. incubator, and allowed to stand for 3 h;
(7) adding culture medium: after standing for 3 h, 15 ml of a prepared culture medium (FBS:DMEM:F12=1:4.5:4.5) was added to the tissue blocks-attached culture bottles in an ultra-clean bench; the 75 T culture bottles with the culture medium were incubated in 5% CO₂, saturated humidity, and 37° C. incubator;
(8) medium-change culture: after culturing for 6 d, the cell growth was observed and recorded on a 7th day, the original culture medium was discarded, and a new culture medium was added with a pipette to the 75 T culture bottles, with 15 ml in each bottle;
(9) specimen passage: after 9 d of culture, the cell growth was observed on a 10th day, and the cells were cultured and passaged in a separation chamber 2; if the cells grew too densely after changing the medium, the cells were handed over in advance; if there was no cell growth after the medium change, a second treatment of the umbilical cord was conducted.

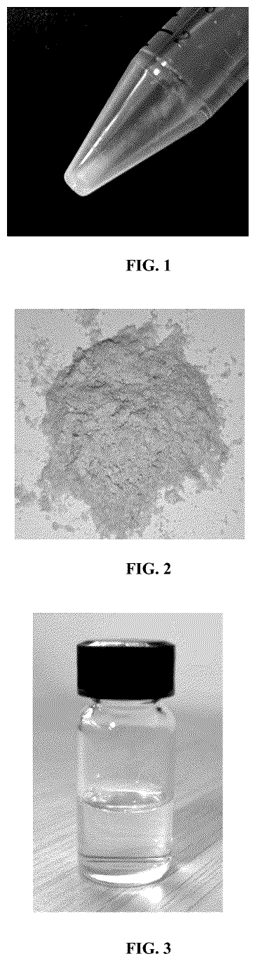

2. Adherent Culture of the Human Umbilical Cord Mesenchymal Stem Cells
(1) the ultra-clean workbench was cleaned and disinfected, the reagents and materials required for the experiment were prepared, an outer surface of the bottle with 75% alcohol was wiped from the bottle cap downward, and put into the ultra-clean workbench immediately;
(2) morphology of the cells observed under the microscope was: spindle-shaped, compact, with swirling growth; when the cell confluence reached 80%, subculture was conducted for expansion and culture;
(3) the culture medium in the culture bottle was aspirated, added with 15 ml to 20 ml of sterile saline or Dhanks for washing, and the sterile saline or Dhanks was discarded into a waste liquid tank; trypsin was added for digestion; the digestion degree of the cells was observed under a microscope, when the cells were curled into a circle, the digestion was terminated; the digested cells were made into a single cell suspension and added to a 50 ml centrifuge tube, 200 μl of the cell suspension was collected for counting before centrifugation, and the centrifugation was conducted at 1,000 r/min for 5 min;
(4) the supernatant was discarded by aspiration, and the cells were inoculated at a density of (1-2)×10⁶/T75 or (2-6)×10⁶/T175 according to the results of cell counting; after inoculation, the barcode number, cell passage number, number of inoculation flasks, operation date and other information were marked on the T75 culture bottles or T175 culture bottles with a marker, and checked; a serum-free basal medium was added, and the cells were incubated in an incubator at 37° C., 5% CO₂ for 24 h to 48 h, and a cell supernatant was collected;
(5) 500 ml of the collected cell supernatant was centrifuged at 500 g/10 min, a supernatant was transferred to a new EP tube, centrifuged at 2,000 g for 15 min at 4° C., a supernatant was transferred to a new EP tube, and centrifuged at 10,000 g for 30 min at 4° C., and filtered with a 0.22 μm filter to obtain a filtrate;
(6) the filtrate was centrifuged at 400 g for 15 min at 4° C. to 6° C., a supernatant was remained, and the precipitate was discarded;
(7) the supernatant obtained in step (6) was centrifuged at 2,500 g for 15 min at 4° C. to 6° C. (Beckman Optima XPN-100 ultracentrifuge), and a supernatant was remained;
(8) the supernatant liquid obtained in step (7) was centrifuged at 12,000 g for 30 min at 4° C. to 6° C., and a supernatant was remained;
(9) the supernatant obtained in step (8) was centrifuged at 150,000 g for 60 min at 4° C. to 6° C. to collect a pellet (FIG. 1);
(10) the pellet obtained in step (9) was resuspended with a PBS buffer, centrifuged at 150,000 g for 60 min at 4° C. to 6° C., a pellet was remained and resuspended with 100 μl to 500 μl of the PBS buffer to obtain a stem cell exosome solution, counted and prepared for a next step.

### Example 2 Preparation of a Freeze-Dried Powder of the Human Umbilical Cord Mesenchymal Stem Cell Exosome

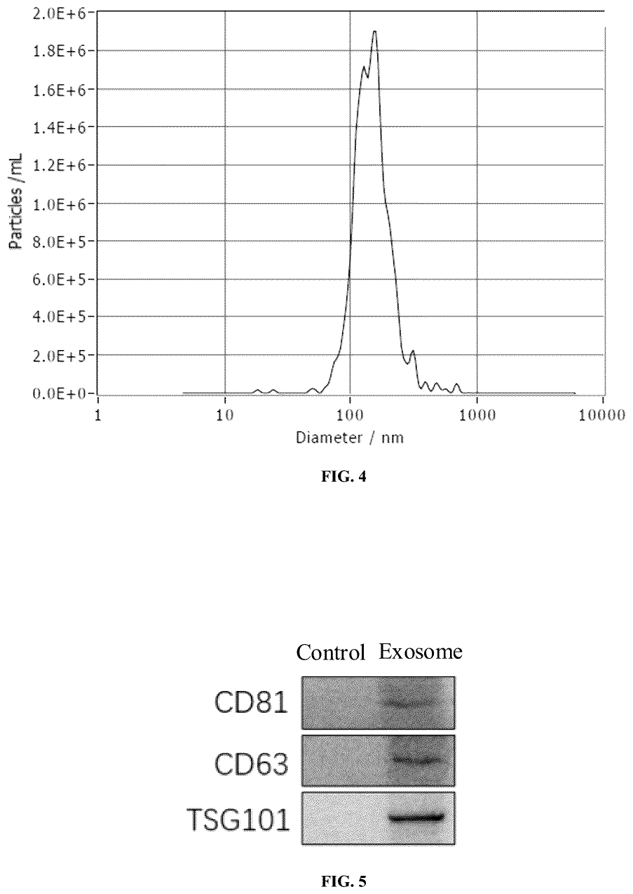

(1) 1 ml of the umbilical cord mesenchymal stem cell exosome solution prepared in Example 1 was diluted and resuspended with 10 ml of a PBS buffer, put in a beaker, and placed in an ultra-low temperature freezer at −86° C. for freezing pretreatment.
(2) The frozen pretreated exosome solution was vacuum freeze-dried at −50° C. (Beijing Sihuan Qihang scientificz-30F/A vacuum freeze dryer) to obtain an umbilical cord mesenchymal stem cell exosome freeze-dried powder (FIG. 2). The exosome freeze-dried powder was stored in a −20° C. refrigerator for 6 months, and then identified by NTA identification, Western blot experimental identification, and transmission electron microscopy detection, where these identification methods were all conducted by conventional identification steps. The identification results showed that the exosome freeze-dried powder stored at 4° C. for 6 months had a relatively stable structure and no decrease in biological efficacy (FIG. 3 to FIG. 5).

### Example 3 Preparation of a Human Umbilical Cord Mesenchymal Stem Cell Exosome-Based Thermosensitive Gel

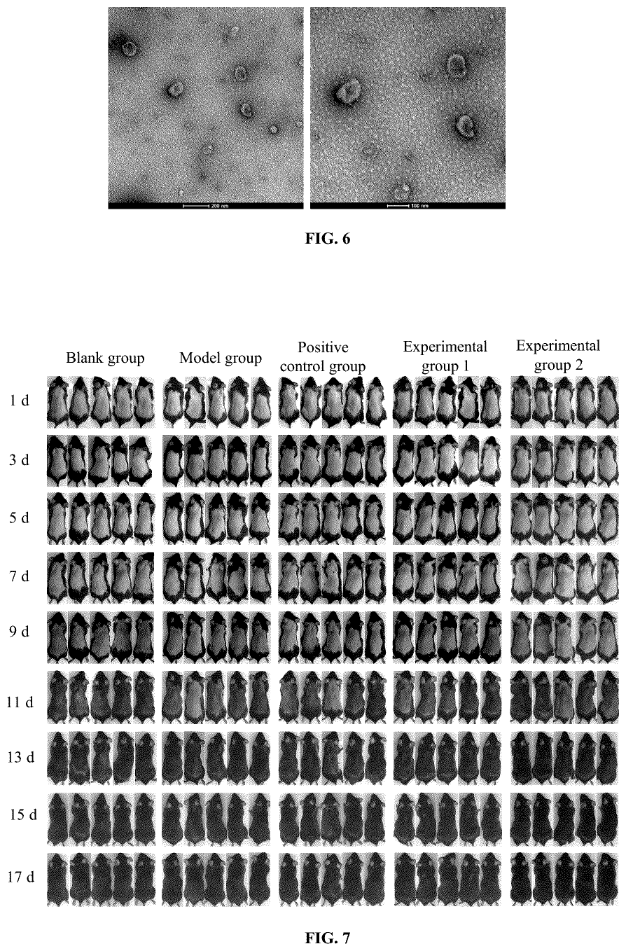

(1) 20% Poloxamer 407 and 4% Poloxamer 188 were added to ultrapure water at a room temperature and allowed to stand for 2 min to 3 min.
(2) The freeze-dried powder prepared in Example 2 was dissolved with tri-distilled water (100 mil ml), added to a gel matrix obtained in step (1) at a mass ratio of 1% and mixed well, and placed in a 4° C. refrigerator for 24 h, to obtain the umbilical cord mesenchymal stem cell exosome-based thermosensitive gel (FIG. 6).

### Example 4 Use of the Human Umbilical Cord Mesenchymal Stem Cell Exosome in an Animal Model with Androgenetic Alopecia

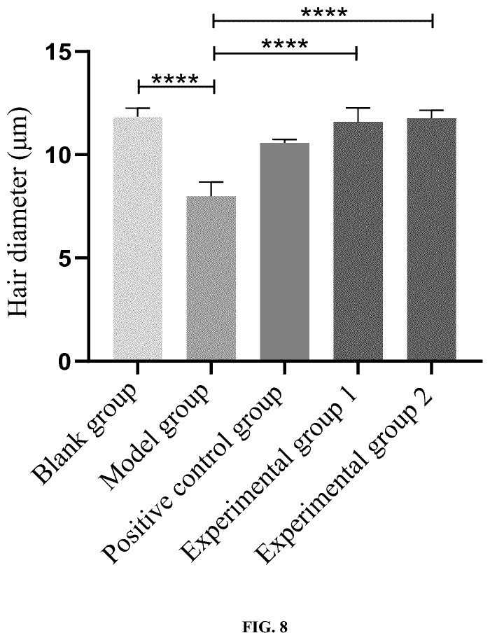

1. 25 6-week-old C57BL6 mice were adaptively fed for one week, and then randomly divided into 5 groups: a blank group, a model group, a positive control group, an experimental group 1, and an experimental group 2. After being anesthetized, the mice in each group were depilated with rosin and paraffin (1:1), with a depilation area of about 2 cm×3 cm. The blank control group did not do any treatment to the depilated mice, and the other mice were intraperitoneally injected with dihydrotestosterone at dosages of 5 times/week and 1 mg/time on the second day of depilation, to establish androgenetic alopecia models, and 5 mice were used as the model group; mice established with androgenetic alopecia models were smeared with a 5% minoxidil solution twice a day, 1 ml each time, for a period of 17 d, as the positive control group; mice established with androgenetic alopecia models were smeared with the thermosensitive gel prepared by the exosome freeze-dried powder (newly-prepared) prepared in Example 2 twice a day, 1 ml each time, for a period of 17 d, as the experimental group 1; mice established with androgenetic alopecia model were smeared with the thermosensitive gel prepared by the exosome freeze-dried powder prepared in Example 2 (refrigerated at 4° C. for 6 months) twice a day, 1 ml each time, for a period of 17 d, as the experimental group 2. During the experiment, photographs were taken every two days to record the hair growth of the mice (FIG. 7). After the experiment, the mice were sacrificed by dislocation; 100 hairs were randomly taken from each mouse, and the diameter and length of the hair were measured; under a 20× microscope, 10 fields of view were randomly selected to count the number of hair follicles; the data was analyzed using Graph Pad Prism software for significant differences, and the difference was statistically significant at the detection level P<0.05 (FIG. 8 to FIG. 10). HE staining was conducted on mouse skin tissues (FIG. 11).

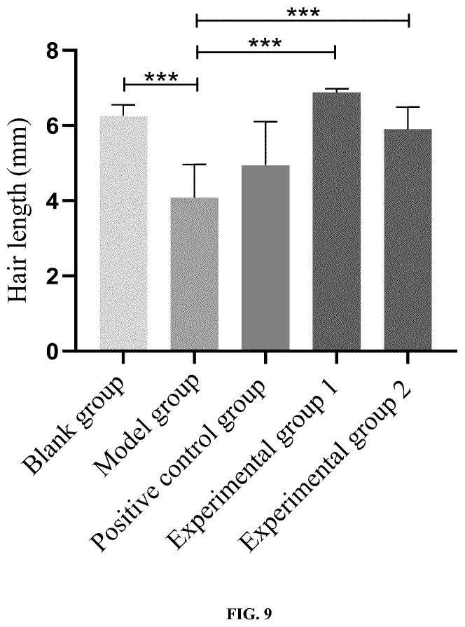

From FIG. 7 combined with FIGS. 8 to 9, the experimental results showed that compared with the blank group, the hair diameter and length of the mice in the model group were significantly reduced *P<0.05; compared with the model group, the hair diameter and length of mice in the experimental group 1 and the experimental group 2 were significantly increased *P<0.05. There was no significant difference between the experimental group 1 and experimental group 2.

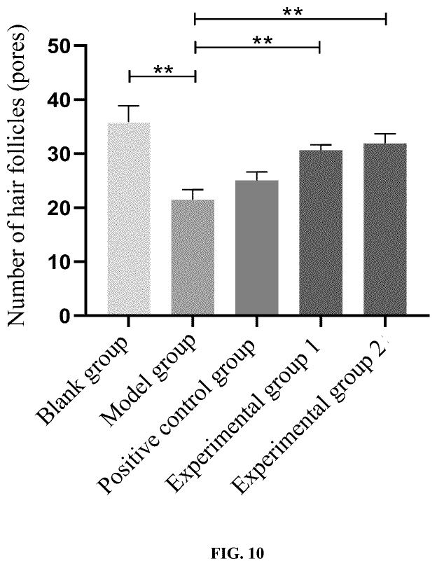

The experimental results in FIGS. 10 to 11 showed that compared with the blank group, the number of hair follicles in the model group was significantly reduced, indicating that the modeling was successful. Compared with the model group, the number of hair follicles in the experimental group 1 and experimental group 2 was significantly increased, indicating that the androgenetic alopecia was effectively ameliorated in mice, and the experimental group 1 and experimental group 2 each had an obvious hair follicle repair effect.

### Example 5 Use of Human Umbilical Cord Mesenchymal Stem Cells in Alopecia Treatment

1. General Information

A total of 60 male patients with seborrheic alopecia treated in the hospital from June 2021 to December 2021 were selected as research subjects, and then divided into a control group and a treatment group by a random number table method, with 30 cases in each group. There was no significant difference in general data such as age, course of disease, and alopecia grade between the two groups of patients (P>0.05), which was comparable. Results were shown in Table 1. This study was approved by the ethics committee of the hospital.

TABLE 1
Comparison of general conditions of two groups of patients

| | No. of | | Average age | Average course of | Alopecia level (case %) | | |
|---|---|---|---|---|---|---|---|
| Group | patients | Sex | (years old) | disease (years) | level II | level III | level IV |
| Control group | 30 | Male | 41.12 ± 5.21 | 2.33 ± 0.28 | 10 (33.33) | 14 (46.66) | 6 (20) |
| Treatment group | 30 | Male | 40.22 ± 6.48 | 2.56 ± 0.77 | 11 (36.66) | 12 (40) | 7 (23.33) |
| Statistics | — | | t = 0.767 | t = 0.341 | | χ² = 0.202 | |
| P value | — | | 0.323 | 0.512 | | 0.894 | |

2. Diagnostic Criteria

Referring to the diagnostic criteria for seborrheic alopecia in “Clinical Dermatology”: (male) the hair on both sides of forehead is gradually sparse and slender, which is gradually extends to the top of head; the anterior hairline is “M”-shaped, and baldness merges with the M shape, leaving hairs only on two temporal and occipital regions.

3. Inclusion and Exclusion Criteria

Inclusion criteria: (1) meeting the above diagnostic criteria of Chinese and Western medicine; (2) 19 to 50 years old; (3) no relevant treatment received within 4 weeks before this visit; (4) complete clinical data capable of cooperating with the research; (5) normal cognition, communication, and audio-visual function; (6) voluntarily participating in this study and signing the informed consent.

Exclusion criteria: (1) alopecia due to physical human factors, drug factors or endocrine disorders; (2) congenital alopecia areata and total alopecia; (3) being combined with scalp injury or bacterial or fungal infection; (4) functional insufficiency in liver and kidney; (5) allergic constitution; (6) mental illness or cognitive impairment; (7) pregnant or breastfeeding women; (8) previous history of hair transplantation surgery.

4. Experimental Method

The control group was given conventional western medicine treatment, male patients were sprayed with 1 ml of 5% minoxidil (Zhejiang Wansheng Pharmaceutical Co., Ltd., product batch number: 20210421) on the affected area, and gently massaged from the affected area to the surrounding area for about 3 minutes, 2 times/day. In the treatment group, 1 ml of exosome freeze-dried powder gel was applied to the affected area, and then gently massaged from the affected area to the surrounding area for about 3 min, 2 times/day. The results were observed after 16 weeks.

5. Determination of Efficacy

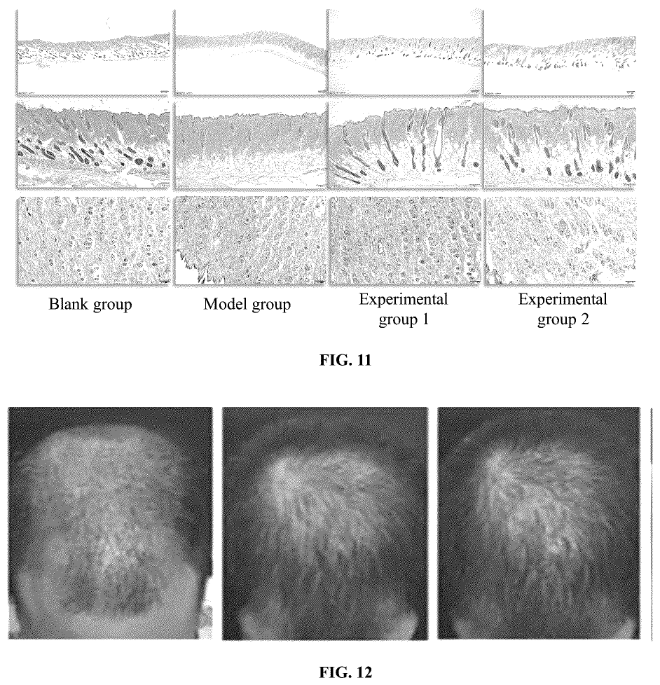

Recovery: alopecia stopped and new hair grew, same as healthy area, with normal sebum secretion ≥95%; significantly effective: alopecia stopped, new hair regenerated ≥70%, hair quality was close to healthy area, and sebum secretion was significantly reduced; effective: alopecia stopped, new hair regenerated ≥30%, syndrome points reduced by 35% to 69%; ineffective: alopecia continued, or new hair regenerated <30%. Total effective rate=cure rate+significantly effective rate+effective rate. The experimental results showed that the therapeutic effect of the treatment group was significantly better than that of the control group (Table 2), and the alopecia area of the treatment group had a significant hair regeneration effect (FIG. 12).

TABLE 2
Comparison of clinical efficacy between two groups of patients (case %)

| | No. of | | Markedly | | | Total |
|---|---|---|---|---|---|---|
| Group | patients | Cured | effective | Effective | Ineffective | efficiency |
| Control group | 30 | 7 (23.33) | 8 (26.66) | 6 (20.00) | 9 (30.00) | 21 (70.00) |
| Treatment group | 30 | 9 (30.00) | 11 (36.66) | 8 (26.66) | 2 (6.66) | 28 (93.33) |
| χ² value | — | — | — | — | — | 4.701 |
| P value | — | — | — | — | — | 0.021 |

The above descriptions are merely preferred implementations of the present disclosure. It should be noted that a person of ordinary skill in the art may further make several improvements and modifications without departing from the principle of the present disclosure, but such improvements and modifications should be deemed as falling within the protection scope of the present disclosure.

1. A method for treating alopecia with a stem cell exosome, wherein the alopecia comprises androgenetic alopecia, seborrheic alopecia, or post-hair transplantation alopecia.

2. The method according to claim 1, wherein the stem cell exosome is one or more selected from the group consisting of a human placenta mesenchymal stem cell exosome, a human umbilical cord mesenchymal stem cell exosome, and a human amniotic fluid mesenchymal stem cell exosome.

3. The method according to claim 1, wherein a preparation method of the stem cell exosome comprises:
culturing mesenchymal stem cells for 24 h to 48 h and collecting a cell supernatant;
conducting centrifugation on the cell supernatant 2 to 4 times and filtering to obtain a filtrate; conducting centrifugation on the filtrate 2 to 4 times, discarding a first pellet, and conducting centrifugation to collect a second pellet; and
resuspending the second pellet with a PBS buffer to obtain a stem cell exosome solution.

4. The method according to claim 3, wherein a medium of the mesenchymal stem cells comprises fetal bovine serum (FBS), Dulbecco's modified eagle medium (DMEM), and F12 at a ratio of (0.5-1.5):(3-6):(3-6).

5. The method according to claim 3, wherein the centrifugation is conducted on the cell supernatant by:
conducting centrifugation on the cell supernatant at 400 g to 600 g for 5 min to 15 min at 3° C. to 5° C. to obtain a first supernatant;
conducting centrifugation on the supernatant I at 1,500 g to 2,500 g for 10 min to 20 min at 3° C. to 5° C. to obtain a second supernatant; and
transferring the second supernatant and conducting centrifugation at 8,000 g to 12,000 g for 30 min at 3° C. to 5° C.

6. The method according to claim 3, wherein the centrifugation is conducted to collect the second pellet II by:
conducting centrifugation at 300 g to 500 g for 10 min to 20 min at 4° C. to 6° C. to obtain a first supernatant I;
conducting centrifugation on the supernatant I at 2,000 g to 3,000 g for 10 min to 20 min at 4° C. to 6° C. to obtain a second supernatant;
conducting centrifugation on the second supernatant at 10,000 g to 15,000 g for 25 min to 35 min at 4° C. to 6° C. to obtain a third supernatant; and
conducting centrifugation on the third supernatant at 125,000 g to 175,000 g for 50 min to 70 min at 4° C. to 6° C. to obtain the second pellet.

7. The method according to claim 1, wherein the second pellet is resuspended with the PBS buffer by: resuspending the second pellet with the PBS buffer, conducting centrifugation at 125,000 g to 175,000 g for 50 min to 70 min at 4° C. to 6° C., discarding a supernatant to obtain a third pellet, and resuspending the third pellet in the PBS buffer.

8. A stem cell exosome freeze-dried powder, prepared by a method comprising pre-freezing a stem cell exosome solution at −80° C. to −95° C., and vacuum freeze-drying at −40° C. to −60° C. to obtain the stem cell exosome freeze-dried powder, wherein the stem cell exosome is prepared by a method comprising:
culturing mesenchymal stem cells for 24 h to 48 h and collecting a cell supernatant; conducting centrifugation on the cell supernatant 2 to 4 times and filtering to obtain a filtrate;
conducting centrifugation on the filtrate 2 to 4 times, discarding a first pellet, and conducting centrifugation to collect a second pellet; and
resuspending the second pellet with a PBS buffer to obtain a stem cell exosome solution.

9. A stem cell exosome-based thermosensitive gel, comprising the following components by weight percentage: 0.5% to 1.5% of the stem cell exosome freeze-dried powder according to claim 8, 10% to 30% of poloxamer 407, and 2% to 6% of poloxamer 188.
